(19) United States
(12) Patent Application Publication
(10) Pub. No.: US 2019/0147986 A1
(43) Pub. Date: May 16, 2019

(54) METHODS FOR ASSESSING NEOADJUVANT THERAPIES

(71) Applicant: Abraxis Bioscience, LLC, Summit, NJ (US)

(72) Inventor: Xiaolong LUO, Belle Mead, NJ (US)

(21) Appl. No.: 16/300,008

(22) PCT Filed: May 17, 2017
(86) PCT No.: PCT/US17/33147

§ 371 Date: Nov. 8, 2018

Related U.S. Application Data
(60) Provisional application No. 62/337,663, filed on May 17, 2016.

(51) Int. Cl. G16H 10/20; A61N 5/10; A61K 31/337

(52) U.S. Cl. CPC G16H 10/20; A61K 31/337; A61N 5/1048

(57) ABSTRACT

Therapies compared in clinical studies can he assessed through the determination of a hazard ratio for long term response between a first a first therapy and a second therapy. Previously, the hazard ratio was determined after a long-term clinical study is conducted to determine the proportion of patients exhibiting a long-term response, such as event free survival or overall survival. Such long-term studies are often cumbersome and expensive. It is has been found that the hazard ratio for long-term response between a first therapy and a second therapy can be determined based on the proportion of patients in a first population of patients receiving the first therapy that exhibit a pathological complete response, the proportion of patients in a second population of patients receiving the second therapy that exhibit the pathological complete response, a patient level effect, and a residual trial level effect. Methods of treating a patient, methods of conducting a clinical trial, and related systems are described.

### CROSS-REFERENCE TO RELATED APPLICATION

This application claims priority benefit to U.S. Provisional Application No. 62/337,663, filed on May 17, 2016, entitled “METHODS FOR ASSESSING NEOADJUVANT THERAPIES.” which is incorporated herein by reference for all purposes.

### FIELD OF THE INVENTION

The present invention relates to methods for assessing therapeutic treatments in a neoadjuvant setting.

### BACKGROUND

In the past decades, advancements in the treatment of early-stage breast cancer in adjuvant and neoadjuvant settings have increased 5 years survival rates to 80% and 90% for patients in England and the United States. Additionally, the three years event free survival rate ranges from 75% to 90% for those with or without pathological complete response, while overall survival rates ranged from 90% to 95%. These relatively low event rates make the ascertainment of long term outcomes difficult even in large clinical trials.

In order to facilitate further development and evaluation of new regimens for treatment of early breast cancer. U.S. Food and Drug Administration (FDA) and the European Medicines Agency (EMA) have published guidance on using pathological complete response as a potential surrogate endpoint for registrational studies (see. e.g., FDA, Guidance for Industry: Pathological Complete Response in Neoadjuvant Treatment of High-Risk Early-Stage Breast Cancer: Use as an Endpoint to Support Accelerated Approval (October 2014)). Prior to the release of the guidance, the FDA granted accelerated approval to pertuzumab based on a multicenter, randomized, open-label phase II trial (NEOSPHERE) that was designed with pathologic complete response as the primary endpoint.

While both FDA and EMA guidance acknowledge that the utility of pathological complete response as the primary endpoint for registrational studies depends on whether it can reasonably likely predict clinical benefit, the quantification of such a condition is not easily described. Cortazar et al., Lancet, vol. 384, pp. 164-172 (2014) conducted a meta-analysis for pathological complete response and long-term outcomes in breast cancer patients as well as breast cancer patients with specific biologic subtypes. However, Cortazar et al. ultimately concluded that pathological complete response as a surrogate endpoint for improved long term response could not be validated.

### BRIEF SUMMARY OF THE INVENTION

Provided herein there is, in some embodiments, a method of treating an individual patient comprising administering a first therapy to the individual patient if a trial level hazard ratio for long-term response between the first therapy and a second therapy is below a predetermined threshold; wherein the trial level hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect.

Also provided herein there is, in some embodiments a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect; and administering the first therapy to a third patient population if the trial level hazard ratio is below a predetermined threshold.

Further provided, in some embodiments, there is a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect.

In some embodiments, the first therapy and the second therapy are neoadjuvant therapies.

In some embodiments, the trial level hazard ratio is determined by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect.

In some embodiments, the patient level effect or the residual trial level effect are determined from a plurality of historical clinical trials. In some embodiments, the patient level effect is based on a hazard ratio between pathological complete response and non pathological complete response for the long-term response in the plurality of historical clinical trials. In some embodiments, the patient level effect is determined using a Cox proportional hazards model. In some embodiments, the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third therapy and a fourth therapy for a long-term response for a given historical clinical trial, i; e^{β} is the patient level effect; π_{i,1} is the proportion of patients in receiving the third therapy that exhibit a pathological complete response in the given historical trial, i; and π_{i,0} is the proportion of patients receiving the fourth therapy that exhibit the pathological complete response in the given historical trial, i. In some embodiments, the third therapy and the fourth therapy are neoadjuvant therapies.

In some embodiments, the long-term response is event free survival. In some embodiments, the long-term response is overall survival.

In some embodiments, the first therapy and the second therapy are cancer therapies. In some embodiments, the first population of patients and the second population of patients have breast cancer. In some embodiments, the first population of patients and the second population of patients have HER2− breast cancer. In some embodiments, the first population of patients and the second population of patients have triple negative breast cancer. In some embodiments, the first population of patients and the second population of patients have HER2+ breast cancer.

In some embodiments, the pathological complete response is ypT0 ypN0 or ypT0/is ypN0.

In some embodiments, the first therapy and the second therapy are breast cancer therapies. In some embodiments, the first therapy and the second therapy comprise administration of a taxane. In some embodiments, the first therapy and the second therapy are followed by surgery or radiation treatment. In some embodiments, the pathological complete response is determined at about the same time as a surgery or radiation treatment.

### DETAILED DESCRIPTION OF THE INVENTION

Provided herein is a method of treating an individual patient comprising administering a first therapy to the individual patient if a trial level hazard ratio for long-term response between the first therapy and a second therapy is below a predetermined threshold; wherein the hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect.

Also provided herein is a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect; and administering the first therapy to a third patient population if the trial level hazard ratio is below a predetermined threshold.

Additionally, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect.

The benefit of a first therapy (such as a test therapy) relative to second therapy (such as a control therapy) can be assessed by determining the relative long-term responses of those therapies. If the first therapy provides better long-term outcomes, the first therapy is generally preferred. These relative long-term responses can be quantified as a hazard ratio between the first therapy and the second therapy. A lower hazard ratio for long-term response indicates that the first therapy provides a better long-term outcome than the second outcome.

Previously, assessing long-term response was determined retrospectively. That is, determining the trial level hazard ratio for long-term response required following populations of patients for a significant duration of time after treatment. In many cases, long-term response is assessed over the course of a decade or longer. Clinical trials are often cumbersome and expensive to conduct for such a long period of time.

The methods described herein allow for a reliable prospective determination of the trial level hazard for long-term response. This prospective determination of the trial level hazard ratio can be made much sooner than previous methods, thereby allowing faster resolution of clinical trials, accelerating the timeline of therapy development, and significantly reducing the cost of drug production. It has been found that the pathological complete response (pCR) can be used as a surrogate metric to reliably assess the trial level hazard ratio for long-term response of a therapy by performing the methods described herein. Determination of pathological complete response (pCR) is made at a much earlier time point than long-term response after the administration of a therapy. In many circumstances, pCR can be determined immediately following a therapy. For example, after administration of a therapy (for example, a neoadjuvant therapy) for the treatment of cancer, a biopsy of the cancer can be performed contemporaneous to a surgery treatment.

As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, stabilized (i.e., not worsening) state of disease, preventing or delaying spread (e.g., metastasis) of disease, preventing or delaying occurrence or recurrence of disease, delay or slowing of disease progression, amelioration of the disease state, and remission (whether partial or total). Also encompassed by “treatment” is a reduction of pathological consequence of a proliferative disease. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “individual” is a mammal, including humans. An individual includes, but is not limited to, human, bovine, horse, feline, canine, rodent, or primate. In some embodiments, the individual is human. The individual (such as human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

It is understood that aspects and embodiments of the invention described herein include “consisting” and/or “consisting essentially of” aspects and embodiments.

As is understood by one skilled in the art, reference to “about” a value or parameter herein includes (and describes) embodiments that are directed to that value or parameter per se. For example, description referring to “about X” includes description of “X”.

The disclosures of all publications, patents, patent applications and published patent applications referred to herein are hereby incorporated herein by reference in their entirety.

### Methods of Treatment

In some embodiments, the methods described herein provide a method for treating an individual patient with a first therapy when the trial-level hazard ratio for long-term response between the first therapy and a second therapy is below a predetermined threshold. The trial-level hazard ratio can be determined based on the proportion of patients in a first population of patients that receive the first therapy (which can be, for example, a test therapy) that exhibit a pathological complete response, the proportion of patients in a second population of patients that receive the second therapy (which can be, for example, a control therapy) that exhibit the pathological complete response, a patient level effect, and a residual trial level effect, as further detailed herein.

In some embodiments, a method of treating an individual patient comprises administering a first therapy to the individual patient if a trial level hazard ratio for long-term response between the first therapy and a second therapy is below a predetermined threshold; wherein the trial level hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect.

The methods allow for prospectively determining a reliable trial-level hazard ratio for long-term response between the first therapy and the second therapy. Thus, the method allows for a first therapy to be selected over a second therapy when the trial-level hazard ratio is below a predetermined threshold without the need to retroactively determine the trial-level hazard ratio through the course of a long-term study.

A trial level hazard ratio between a first therapy and a second therapy of less than 1 indicates a more favorable long-term response for the first therapy than the second therapy. In some embodiments, the predetermined threshold is about 1 or less, about 0.95 or less, about 0.9 or less, about 0.85 or less, about 0.80 or less, about 0.75 or less, about 0.70 or less, about 0.65 or less, about 0.55 or less, about 0.50 or less, about 0.45 or less, about 0.40 or less, about 0.35 or less, about 0.30 or less, about 0.25 or less, about 0.20 or less, about 0.15 or less, or about 0.10 or less. The predetermined threshold can be determined based on any number of factors, such as therapy cost, patient compliance, therapy side effects, ease of administration, patient comfort, etc. For example, a significantly higher cost for the first therapy than the second therapy may justify a lower predetermined threshold for the trial-level hazard ratio for administration of the first therapy to the individual patient.

In some embodiments, the individual has cancer. The first therapy is administered to the individual patient to treat the patient. In some embodiments, the individual has cancer and, and the first therapy is used to treat the cancer. In some embodiments, the individual patient has breast cancer. In some embodiments, the cancer is bladder cancer, brain cancer, breast cancer, carcinoid tumor, cervical cancer, colorectal cancer, endometrial cancer, esophageal cancer, gastric cancer, hepatocellular cancer, laryngeal cancer, lip cancer, oral cancer, lung cancer, melanoma, ovarian cancer, pancreatic cancer, pharyngeal cancer, prostate cancer, renal cancer, retinoblastoma, testicular cancer, or thyroid cancer.

In some embodiments, the cancer is identified by a particular biological subtype. For example, in some embodiments, the breast cancer is HER2+ breast cancer. In some embodiments, the breast cancer is HER2− breast cancer. In some embodiments, the breast cancer is estrogen receptor positive (ER+) breast cancer. In some embodiments, the breast cancer is estrogen receptor negative (ER−) breast cancer. In some embodiments, the breast cancer is progesterone receptor positive (PR+) breast cancer. In some embodiments, the breast cancer is progesterone receptor negative (PR−) breast cancer. In some embodiments, the breast cancer is triple negative (HER2−, ER−, PR−) breast cancer (TNBC).

In some embodiments, the cancer is identified by a particular tumor stage. For example, in some embodiments, the tumor stage is T1, T2, T3, T4a, T4b, T4c, or T4d. In some embodiments, the cancer is identified by a particular node stage. For example, in some embodiments, the node stage is N1, N2, or N3.

In some embodiments, the therapies (e.g., the first therapy and the second therapy) are neoadjuvant therapies, i.e., the therapies are carried out before the primary/definitive therapy. In some embodiments, the primary/definitive therapy is a surgery (for example, a lumpectomy or mastectomy in the circumstance of breast cancer). In some embodiments, the primary/definitive therapy is radiation therapy. In some embodiments, the therapies are adjuvant therapies. In some embodiments, the individual has previously been treated. In some embodiments, the individual has not previously been treated. In some embodiments, the treatment is a first line therapy.

In some embodiments, the first therapy or the second therapy comprises administration of an effective amount of a taxane (such as paclitaxel, docetaxel, or ortataxel). In some embodiments, the first therapy or the second therapy further comprises administration of an effective amount of at least one other chemotherapeutic agent (such as an anthracycline or a cyclophosphamide) and/or an antibody.

A pathological complete response can be determined for a cancer with any stringency, for example in accordance with the TNM staging system used by the Union for International cancer Control (UICC). In some embodiments, the pathological complete response is the absence of invasive or in situ residual cancer in the tissue (such as breast tissue) or axillary lymph nodes (ypT0 ypN0). In some embodiments, the pathological complete response is the absence of invasive residual cancer in tissue (such as breast tissue) or axillary lymph nodes, irrespective of the presence or absence of in situ residual cancer (ypT0/is ypN0). In some embodiments, the pathological complete response is the absence of invasive or in situ residual cancer in the tissue (such as breast tissue), irrespective of the presence or absence of cancer in axillary lymph nodes (ypT0). In some embodiments, the pathological complete response is the absence of invasive residual cancer in the tissue (such as breast tissue), irrespective of the presence or absence of cancer in axillary lymph nodes or in situ residual cancer (ypT0/is).

Pathological complete response is determined after the administration of the therapy. For example, in some embodiments, the cancer is biopsied after the administration of the therapy. In some embodiments, the pathological complete response is determined about the same time as administration of a determinative therapy (such as surgery) after the administration of a neoadjuvant therapy. For example, in some embodiments, the surgery is performed on an individual with cancer to recess a portion of the cancer, after which a specimen is analyzed for pathology. The pathological complete response can be assessed from the specimen.

The proportion of patients exhibiting the pathological complete response in a patient population is determined by dividing the number of individual patients exhibiting the pathological complete response by the total number of individual patients in the patient population. In some embodiments, patients with a common identifier are randomly distributed into a first patient population and a second patient population. The common identifier can be, for example, a particular disease, such as a particular cancer (e.g., type of cancer (breast cancer, prostate cancer, etc.), biological subtype (e.g., HER2+ breast cancer, triple negative breast cancer, etc.), tumor stage, nodal stage etc.). The patients in the first patient population are administered a first therapy (such as a test therapy), and the patients in the second patient population are administered a second therapy (such as a control therapy). After completion of the therapy, pathological complete response is determined for the patients in the first patient population and the second patient population. The proportion of patients in the first patient population exhibiting the pathological complete response and the proportion of patients in the second patient population exhibiting the pathological complete response can thusly be obtained. In some embodiments, the proportion of patients in the first patient population exhibiting the pathological complete response and the proportion of patients in the second patient population exhibiting the pathological complete response is obtained from a prior clinical study.

The patient level effect and the residual trial level effect can be determined from one or more historical clinical trials, as further described herein.

In some embodiments, there is provided a method of treating an individual patient comprising administering a first therapy to the individual patient if a trial level hazard ratio for long-term response between the first therapy and a second therapy is below a predetermined threshold (such as about 1 or less); wherein the trial level hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a method of treating an individual patient with cancer comprising administering a first cancer therapy to the individual patient if a trial level hazard ratio for long-term response between the first cancer therapy and a second cancer therapy is below a predetermined threshold (such as about 1 or less); wherein the trial level hazard ratio is determined by obtaining a proportion of patients in a first population of patients receiving the first cancer therapy that exhibit a pathological complete response; obtaining a proportion of patients in a second population of patients receiving the second cancer therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a method of treating an individual patient comprising administering a first neoadjuvant therapy to the individual patient if a trial level hazard ratio for long-term response between the first neoadjuvant therapy and a second neoadjuvant therapy is below a predetermined threshold (such as about 1 or less); wherein the trial level hazard ratio is determined by obtaining a proportion of patients in a first population of patients receiving the first neoadjuvant therapy that exhibit a pathological complete response; obtaining a proportion of patients in a second population of patients receiving the second neoadjuvant therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a method of treating an individual patient with cancer comprising administering a first neoadjuvant cancer therapy to the individual patient if a trial level hazard ratio for long-term response between the first neoadjuvant cancer therapy and a second neoadjuvant cancer therapy is below a predetermined threshold (such as about 1 or less); wherein the trial level hazard ratio is determined by obtaining a proportion of patients in a first population of patients receiving the first neoadjuvant cancer therapy that exhibit a pathological complete response; obtaining a proportion of patients in a second population of patients receiving the second neoadjuvant cancer therapy that exhibit the pathological complete response; obtaining a patient level effect and a residual trial level effect; and determining the trial level hazard ratio based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a method of treating an individual patient comprising administering a first therapy to the individual patient if a trial level hazard ratio for long-term response between the first therapy and a second therapy is below a predetermined threshold (such as about 1 or less); wherein the trial level hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a method of treating an individual patient with cancer comprising administering a first cancer therapy to the individual patient if a trial level hazard ratio for long-term response between the first cancer therapy and a second cancer therapy is below a predetermined threshold (such as about 1 or less); wherein the trial level hazard ratio is determined by obtaining a proportion of patients in a first population of patients receiving the first cancer therapy that exhibit a pathological complete response; obtaining a proportion of patients in a second population of patients receiving the second cancer therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a method of treating an individual patient comprising administering a first neoadjuvant therapy to the individual patient if a trial level hazard ratio for long-term response between the first neoadjuvant therapy and a second neoadjuvant therapy is below a predetermined threshold (such as about 1 or less); wherein the trial level hazard ratio is determined by obtaining a proportion of patients in a first population of patients receiving the first neoadjuvant therapy that exhibit a pathological complete response; obtaining a proportion of patients in a second population of patients receiving the second neoadjuvant therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a method of treating an individual patient with cancer comprising administering a first neoadjuvant cancer therapy to the individual patient if a trial level hazard ratio for long-term response between the first neoadjuvant cancer therapy and a second neoadjuvant cancer therapy is below a predetermined threshold (such as about 1 or less); wherein the trial level hazard ratio is determined by obtaining a proportion of patients in a first population of patients receiving the first neoadjuvant cancer therapy that exhibit a pathological complete response; obtaining a proportion of patients in a second population of patients receiving the second neoadjuvant cancer therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a method of treating an individual patient comprising administering a first therapy to the individual patient if a trial level hazard ratio for long-term response between the first therapy and a second therapy is below a predetermined threshold (such as about 1 or less); wherein the trial level hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a method of treating an individual patient with cancer comprising administering a first cancer therapy to the individual patient if a trial level hazard ratio for long-term response between the first cancer therapy and a second cancer therapy is below a predetermined threshold (such as about 1 or less); wherein the trial level hazard ratio is determined by obtaining a proportion of patients in a first population of patients receiving the first cancer therapy that exhibit a pathological complete response; obtaining a proportion of patients in a second population of patients receiving the second cancer therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a method of treating an individual patient comprising administering a first neoadjuvant therapy to the individual patient if a trial level hazard ratio for long-term response between the first neoadjuvant therapy and a second neoadjuvant therapy is below a predetermined threshold (such as about 1 or less); wherein the trial level hazard ratio is determined by obtaining a proportion of patients in a first population of patients receiving the first neoadjuvant therapy that exhibit a pathological complete response; obtaining a proportion of patients in a second population of patients receiving the second neoadjuvant therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials.

In some embodiments, there is provided a method of treating an individual patient with cancer comprising administering a first neoadjuvant cancer therapy to the individual patient if a trial level hazard ratio for long-term response between the first neoadjuvant cancer therapy and a second neoadjuvant cancer therapy is below a predetermined threshold (such as about 1 or less); wherein the trial level hazard ratio is determined by obtaining a proportion of patients in a first population of patients receiving the first neoadjuvant cancer therapy that exhibit a pathological complete response; obtaining a proportion of patients in a second population of patients receiving the second neoadjuvant cancer therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a method of treating an individual patient comprising administering a first therapy to the individual patient if a trial level hazard ratio for long-term response between the first therapy and a second therapy is below a predetermined threshold (such as about 1 or less); wherein the trial level hazard ratio is determined by obtaining a proportion of patients in a first population of patients receiving the first therapy that exhibit a pathological complete response; obtaining a proportion of patients in a second population of patients receiving the second therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials, and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third therapy and a fourth therapy for a long-term response for a given historical clinical trial, i; e^{β} is the patient level effect; π_{i,1} is the proportion of patients in receiving the third therapy that exhibit a pathological complete response in the given historical trial, i; and π_{i,0} is the proportion of patients in receiving the fourth therapy that exhibit the pathological complete response in the given historical trial, i. In some embodiments, the long-term response is event free survival. In some embodiments, the long-term response is overall survival.

In some embodiments, there is provided a method of treating an individual patient with cancer comprising administering a first cancer therapy to the individual patient if a trial level hazard ratio for long-term response between the first cancer therapy and a second cancer therapy is below a predetermined threshold (such as about 1 or less); wherein the trial level hazard ratio is determined by obtaining a proportion of patients in a first population of patients receiving the first cancer therapy that exhibit a pathological complete response; obtaining a proportion of patients in a second population of patients receiving the second cancer therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials, and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third cancer therapy and a fourth cancer therapy for a long-term response for a given historical clinical trial, i; e^{β} is the patient level effect; π_{i,1} is the proportion of patients in receiving the third cancer therapy that exhibit a pathological complete response in the given historical trial, i; and π_{i,0} is the proportion of patients in receiving the fourth cancer therapy that exhibit the pathological complete response in the given historical trial, i. In some embodiments, the long-term response is event free survival. In some embodiments, the long-term response is overall survival.

In some embodiments, there is provided a method of treating an individual patient comprising administering a first neoadjuvant therapy to the individual patient if a trial level hazard ratio for long-term response between the first neoadjuvant therapy and a second neoadjuvant therapy is below a predetermined threshold (such as about 1 or less); wherein the trial level hazard ratio is determined by obtaining a proportion of patients in a first population of patients receiving the first neoadjuvant therapy that exhibit a pathological complete response; obtaining a proportion of patients in a second population of patients receiving the second neoadjuvant therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials, and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third neoadjuvant therapy and a fourth neoadjuvant therapy for a long-term response for a given historical clinical trial, i; e^{β} is the patient level effect; π_{i,1} is the proportion of patients in receiving the third neoadjuvant therapy that exhibit a pathological complete response in the given historical trial, i; and π_{i,0} is the proportion of patients in receiving the fourth neoadjuvant therapy that exhibit the pathological complete response in the given historical trial, i. In some embodiments, the long-term response is event free survival. In some embodiments, the long-term response is overall survival.

In some embodiments, there is provided a method of treating an individual patient with cancer comprising administering a first neoadjuvant cancer therapy to the individual patient if a trial level hazard ratio for long-term response between the first neoadjuvant cancer therapy and a second neoadjuvant cancer therapy is below a predetermined threshold (such as about 1 or less); wherein the trial level hazard ratio is determined by obtaining a proportion of patients in a first population of patients receiving the first neoadjuvant cancer therapy that exhibit a pathological complete response; obtaining a proportion of patients in a second population of patients receiving the second neoadjuvant cancer therapy that exhibit the pathological complete response; obtaining a patient level effect and a residual trial level effect; and determining the trial level hazard ratio by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials, and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third neoadjuvant cancer therapy and a fourth neoadjuvant cancer therapy for a long-term response for a given historical clinical trial, i; e^{β} is the patient level effect; π_{i,1} is the proportion of patients in receiving the third neoadjuvant cancer therapy that exhibit a pathological complete response in the given historical trial, i; and π_{i,0} is the proportion of patients in receiving the fourth neoadjuvant cancer therapy that exhibit the pathological complete response in the given historical trial, i. In some embodiments, the long-term response is event free survival. In some embodiments, the long-term response is overall survival.

### Methods of Conducting a Therapy Trial

In some embodiments, there is provided a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect; and administering the first therapy to a third patient population if the trial level hazard ratio is below a predetermined threshold. In some embodiments, the second therapy is administered to a fourth patient population. In some embodiments, a retrospective trial level hazard ratio between the first therapy and the second therapy is determined based on the long-term response of the administration of the first therapy to the third patient population and administration of the second therapy to the fourth patient population.

In some embodiments, the third patient population or the fourth patient population is subject to a long-term study. In some embodiments, the long-term study last 1 or more years, 2 or more years, 3 or more years, 4 or more years, 5 or more years, 6 or more years, 7 or more years, 8 or more years, 9 or more years, 10 or more years, 11 or more years, 12 or more years, 13 or more years, 14 or more years, or 15 or more years. In some embodiments, the long-term response of patients in the third patient population is recorded. In some embodiments, the long-term response is overall survival, event free survival, disease free survival, or progression free survival.

Prospectively determining the trial-level hazard ratio can be used as a factor in deciding whether to continue a longer-term study. For example, in some embodiments, the first therapy is not administered to the third patient population if the trial level hazard ratio is above the predetermined threshold.

In some embodiments, the predetermined threshold is about 1 or less, about 0.95 or less, about 0.9 or less, about 0.85 or less, about 0.80 or less, about 0.75 or less, about 0.70 or less, about 0.65 or less, about 0.55 or less, about 0.50 or less, about 0.45 or less, about 0.40 or less, about 0.35 or less, about 0.30 or less, about 0.25 or less, about 0.20 or less, about 0.15 or less, or about 0.10 or less.

In some embodiments, the patients in the first patient population, the second population, and the third patient population have cancer. In some embodiments, the patients in the first patient population, the second population, and the third patient population have breast cancer. In some embodiments, the cancer is bladder cancer, brain cancer, breast cancer, carcinoid tumor, cervical cancer, colorectal cancer, endometrial cancer, esophageal cancer, gastric cancer, hepatocellular cancer, laryngeal cancer, lip cancer, oral cancer, lung cancer, melanoma, ovarian cancer, pancreatic cancer, pharyngeal cancer, prostate cancer, renal cancer, retinoblastoma, testicular cancer, or thyroid cancer.

In some embodiments, the cancer is identified by a particular biological subtype. For example, in some embodiments, the breast cancer is HER2+ breast cancer. In some embodiments, the breast cancer is HER2− breast cancer. In some embodiments, the breast cancer is estrogen receptor positive (ER+) breast cancer. In some embodiments, the breast cancer is estrogen receptor negative (ER−) breast cancer. In some embodiments, the breast cancer is progesterone receptor positive (PR+) breast cancer. In some embodiments, the breast cancer is progesterone receptor negative (PR−) breast cancer. In some embodiments, the breast cancer is triple negative (HER2−, ER−, PR−) breast cancer (TNBC).

In some embodiments, the cancer is identified by a particular tumor stage. For example, in some embodiments, the tumor stage is T1, T2, T3, T4a, T4b, T4c, or T4d. In some embodiments, the cancer is identified by a particular node stage. For example, in some embodiments, the node stage is N1, N2, or N3.

In some embodiments, the therapies (e.g., the first therapy and the second therapy) are neoadjuvant therapies, i.e., the therapies are carried out before the primary/definitive therapy. In some embodiments, the primary/definitive therapy is a surgery (for example, a lumpectomy or mastectomy in the circumstance of breast cancer). In some embodiments, the primary/definitive therapy is radiation therapy. In some embodiments, the therapies are adjuvant therapies. In some embodiments, the individual has previously been treated. In some embodiments, the individual has not previously been treated. In some embodiments, the treatment is a first line therapy.

In some embodiments, the first therapy or the second therapy comprises administration of an effective amount of a taxane (such as paclitaxel, docetaxel, or ortataxel). In some embodiments, the first therapy or the second therapy further comprises administration of an effective amount of at least one other chemotherapeutic agent (such as an anthracycline or a cyclophosphamide) and/or an antibody.

A pathological complete response can be determined for a cancer with any stringency, for example in accordance with the TNM staging system used by the Union for International cancer Control (UICC). In some embodiments, the pathological complete response is the absence of invasive or in situ residual cancer in the tissue (such as breast tissue) or axillary lymph nodes (ypT0 ypN0). In some embodiments, the pathological complete response is the absence of invasive residual cancer in tissue (such as breast tissue) or axillary lymph nodes, irrespective of the presence or absence of in situ residual cancer (ypT0/is ypN0). In some embodiments, the pathological complete response is the absence invasive or in situ residual cancer in the tissue (such as breast tissue), irrespective of the presence or absence of cancer in axillary lymph nodes (ypT0). In some embodiments, the pathological complete response is the absence of invasive residual cancer in the tissue (such as breast tissue), irrespective of the presence or absence of cancer in axillary lymph nodes or in situ residual cancer (ypT0/is).

Pathological complete response is determined after the administration of the therapy. For example, in some embodiments, the cancer is biopsied after the administration of the therapy. In some embodiments, the pathological complete response is determined about the same time as administration of a determinative therapy (such as surgery) after the administration of a neoadjuvant therapy. For example, in some embodiments, the surgery is performed on an individual with cancer to recess a portion of the cancer, after which a specimen is analyzed for pathology. The pathological complete response can be assessed from the specimen.

The proportion of patients exhibiting the pathological complete response in a patient population is determined by dividing the number of individual patients exhibiting the pathological complete response by the total number of individual patients in the patient population. In some embodiments, patients with a common identifier are randomly distributed into a first patient population and a second patient population. The common identifier can be, for example, a particular disease, such as a particular cancer (e.g., type of cancer (breast cancer, prostate cancer, etc.), biological subtype (e.g., HER2+ breast cancer, triple negative breast cancer, etc.), tumor stage, nodal stage etc.). The patients in the first patient population are administered a first therapy (such as a test therapy), and the patients in the second patient population are administered a second therapy (such as a control therapy). After completion of the therapy, pathological complete response is determined for the patients in the first patient population and the second patient population. The proportion of patients in the first patient population exhibiting the pathological complete response and the proportion of patients in the second patient population exhibiting the pathological complete response can thusly be obtained. In some embodiments, the proportion of patients in the first patient population exhibiting the pathological complete response and the proportion of patients in the second patient population exhibiting the pathological complete response is obtained from a prior clinical study.

The patient level effect and the residual trial level effect can be determined from one or more historical clinical trials, as further described herein.

In some embodiments, there is provided a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect; and administering the first therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a cancer therapy trial comprising obtaining a proportion of patients in a first patient population receiving a first cancer therapy that exhibit a pathological complete response; obtaining a proportion of patients in a second patient population receiving a second cancer therapy that exhibit the pathological complete response; obtaining a patient level effect and a residual trial level effect; determining a trial level hazard ratio for long-term response between the first cancer therapy and the second cancer therapy based on the proportion of patients in the first population of patients that exhibit the pathological complete response, the proportion of patients in the second population of patients that exhibit the pathological complete response, the patient level effect, and the residual trial level effect; and administering the first cancer therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a neoadjuvant therapy trial comprising obtaining a proportion of patients in a first patient population receiving a first neoadjuvant therapy that exhibit a pathological complete response; obtaining a proportion of patients in a second patient population receiving a second neoadjuvant therapy that exhibit the pathological complete response; obtaining a patient level effect and a residual trial level effect; determining a trial level hazard ratio for long-term response between the first neoadjuvant therapy and the second neoadjuvant therapy based on the proportion of patients in the first population of patients that exhibit the pathological complete response, the proportion of patients in the second population of patients that exhibit the pathological complete response, the patient level effect, and the residual trial level effect; and administering the first neoadjuvant therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a neoadjuvant cancer therapy trial comprising obtaining a proportion of patients in a first patient population receiving a first neoadjuvant cancer therapy that exhibit a pathological complete response; obtaining a proportion of patients in a second patient population receiving a second neoadjuvant cancer therapy that exhibit the pathological complete response; obtaining a patient level effect and a residual trial level effect; determining a trial level hazard ratio for long-term response between the first neoadjuvant cancer therapy and the second neoadjuvant cancer therapy based on the proportion of patients in the first population of patients that exhibit the pathological complete response, the proportion of patients in the second population of patients that exhibit the pathological complete response, the patient level effect, and the residual trial level effect; and administering the first neoadjuvant cancer therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a cancer therapy trial comprising obtaining a proportion of patients in a first patient population receiving a first cancer therapy that exhibit a pathological complete response; obtaining a proportion of patients in a second patient population receiving a second cancer therapy that exhibit the pathological complete response; obtaining a patient level effect and a residual trial level effect; determining a trial level hazard ratio for long-term response between the first cancer therapy and the second cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first cancer therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a neoadjuvant therapy trial comprising obtaining a proportion of patients in a first patient population receiving a first neoadjuvant therapy that exhibit a pathological complete response; obtaining a proportion of patients in a second patient population receiving a second neoadjuvant therapy that exhibit the pathological complete response; obtaining a patient level effect and a residual trial level effect; determining a trial level hazard ratio for long-term response between the first neoadjuvant therapy and the second neoadjuvant therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first neoadjuvant therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a neoadjuvant cancer therapy trial comprising obtaining a proportion of patients in a first patient population receiving a first neoadjuvant cancer therapy that exhibit a pathological complete response; obtaining a proportion of patients in a second patient population receiving a second neoadjuvant cancer therapy that exhibit the pathological complete response; obtaining a patient level effect and a residual trial level effect; determining a trial level hazard ratio for long-term response between the first neoadjuvant cancer therapy and the second neoadjuvant cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first neoadjuvant cancer therapy to a third patient population if the trial level hazard ratio is below a predetermined threshold (such as about 1 or less). In some embodiments, the long-term response is event free survival. In some embodiments, the long-term response is overall survival.

In some embodiments, there is provided a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first therapy to a third patient population if the trial level hazard ratio is below a predetermined threshold (such as about 1 or less); wherein the patient level effect or the residual trial level effect are determined from a plurality of historical clinical trials. In some embodiments, the long-term response is event free survival. In some embodiments, the long-term response is overall survival.

In some embodiments, there is provided a method of conducting a cancer therapy trial comprising obtaining a proportion of patients in a first patient population receiving a first cancer therapy that exhibit a pathological complete response; obtaining a proportion of patients in a second patient population receiving a second cancer therapy that exhibit the pathological complete response; obtaining a patient level effect and a residual trial level effect; determining a trial level hazard ratio for long-term response between the first cancer therapy and the second cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first cancer therapy to a third patient population if the trial level hazard ratio is below a predetermined threshold (such as about 1 or less); wherein the patient level effect or the residual trial level effect are determined from a plurality of historical clinical trials. In some embodiments, the long-term response is event free survival. In some embodiments, the long-term response is overall survival.

In some embodiments, there is provided a method of conducting a neoadjuvant therapy trial comprising obtaining a proportion of patients in a first patient population receiving a first neoadjuvant therapy that exhibit a pathological complete response; obtaining a proportion of patients in a second patient population receiving a second neoadjuvant therapy that exhibit the pathological complete response; obtaining a patient level effect and a residual trial level effect; determining a trial level hazard ratio for long-term response between the first neoadjuvant therapy and the second neoadjuvant therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first neoadjuvant therapy to a third patient population if the trial level hazard ratio is below a predetermined threshold (such as about 1 or less); wherein the patient level effect or the residual trial level effect are determined from a plurality of historical clinical trials. In some embodiments, the long-term response is event free survival. In some embodiments, the long-term response is overall survival.

In some embodiments, there is provided a method of conducting a neoadjuvant cancer therapy trial comprising obtaining a proportion of patients in a first patient population receiving a first neoadjuvant cancer therapy that exhibit a pathological complete response; obtaining a proportion of patients in a second patient population receiving a second neoadjuvant cancer therapy that exhibit the pathological complete response; obtaining a patient level effect and a residual trial level effect; determining a trial level hazard ratio for long-term response between the first neoadjuvant cancer therapy and the second neoadjuvant cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first neoadjuvant cancer therapy to a third patient population if the trial level hazard ratio is below a predetermined threshold (such as about 1 or less); wherein the patient level effect or the residual trial level effect are determined from a plurality of historical clinical trials. In some embodiments, the long-term response is event free survival. In some embodiments, the long-term response is overall survival.

In some embodiments, there is provided a method of conducting a therapy trial comprising obtaining a proportion of patients in a first patient population receiving a first therapy that exhibit a pathological complete response; obtaining a proportion of patients in a second patient population receiving a second therapy that exhibit the pathological complete response; obtaining a patient level effect and a residual trial level effect; determining a trial level hazard ratio for long-term response between the first therapy and the second therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first therapy to a third patient population if the trial level hazard ratio is below a predetermined threshold (such as about 1 or less); wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{{β}_{i}} - 1 ) {π}_{i , 0}} ,$$

wherein λᵢ is the hazard ratio between a third therapy and a fourth therapy for a long-term response for a given historical clinical trial, i; e^{β} is the patient level effect; π_{i,1} is the proportion of patients in receiving the third therapy that exhibit a pathological complete response in the given historical trial, i; and π_{i,0} is the proportion of patients in receiving the fourth therapy that exhibit the pathological complete response in the given historical trial, i. In some embodiments, the long-term response is event free survival. In some embodiments, the long-term response is overall survival.

In some embodiments, there is provided a method of conducting a cancer therapy trial comprising obtaining a proportion of patients in a first patient population receiving a first cancer therapy that exhibit a pathological complete response; obtaining a proportion of patients in a second patient population receiving a second cancer therapy that exhibit the pathological complete response; obtaining a patient level effect and a residual trial level effect; determining a trial level hazard ratio for long-term response between the first cancer therapy and the second cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first cancer therapy to a third patient population if the trial level hazard ratio is below a predetermined threshold (such as about 1 or less); wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third cancer therapy and a fourth cancer therapy for a long-term response for a given historical clinical trial, i; e^{β} is the patient level effect; π_{i,1} is the proportion of patients in receiving the third cancer therapy that exhibit a pathological complete response in the given historical trial, i; and π_{i,0} is the proportion of patients in receiving the fourth cancer therapy that exhibit the pathological complete response in the given historical trial, i. In some embodiments, the long-term response is event free survival. In some embodiments, the long-term response is overall survival.

In some embodiments, there is provided a method of conducting a neoadjuvant therapy trial comprising obtaining a proportion of patients in a first patient population receiving a first neoadjuvant therapy that exhibit a pathological complete response; obtaining a proportion of patients in a second patient population receiving a second neoadjuvant therapy that exhibit the pathological complete response; obtaining a patient level effect and a residual trial level effect; determining a trial level hazard ratio for long-term response between the first neoadjuvant therapy and the second neoadjuvant therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first neoadjuvant therapy to a third patient population if the trial level hazard ratio is below a predetermined threshold (such as about 1 or less); wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third neoadjuvant therapy and a fourth neoadjuvant therapy for a long-term response for a given historical clinical trial, i; e^{β} is the patient level effect; π_{i,1} is the proportion of patients in receiving the third neoadjuvant therapy that exhibit a pathological complete response in the given historical trial, i; and π_{i,0} is the proportion of patients in receiving the fourth neoadjuvant therapy that exhibit the pathological complete response in the given historical trial, i. In some embodiments, the long-term response is event free survival. In some embodiments, the long-term response is overall survival.

In some embodiments, there is provided a method of conducting a neoadjuvant cancer therapy trial comprising obtaining a proportion of patients in a first patient population receiving a first neoadjuvant cancer therapy that exhibit a pathological complete response; obtaining a proportion of patients in a second patient population receiving a second neoadjuvant cancer therapy that exhibit the pathological complete response; obtaining a patient level effect and a residual trial level effect; determining a trial level hazard ratio for long-term response between the first neoadjuvant cancer therapy and the second neoadjuvant cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect; and administering the first neoadjuvant cancer therapy to a third patient population if the trial level hazard ratio is below a predetermined threshold (such as about 1 or less); wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third neoadjuvant cancer therapy and a fourth neoadjuvant cancer therapy for a long-term response for a given historical clinical trial, i; e^{β} is the patient level effect; π_{i,1} is the proportion of patients in receiving the third neoadjuvant cancer therapy that exhibit a pathological complete response in the given historical trial, i; and π_{i,0} is the proportion of patients in receiving the fourth neoadjuvant cancer therapy that exhibit the pathological complete response in the given historical trial, i. In some embodiments, the long-term response is event free survival. In some embodiments, the long-term response is overall survival.

### Computing Systems

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect.

An exemplary computing system configured to perform any one of the processes described herein, including the various exemplary processes determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect may include a processor, memory, storage, and input/output devices (e.g., monitor, keyboard, disk drive. Internet connection, etc.). However, the computing system may include circuitry or other specialized hardware for carrying out some or all aspects of the processes. In some operational settings, the computing system may be configured as a system that includes one or more units, each of which is configured to carry out some aspects of the processes either in software, hardware, or some combination thereof.

The computing system can comprise a number of components that may be used to perform the processes described herein. The main system can include a motherboard having an input/output (“I/O”) section, one or more central processing units (“CPU”), and a memory section, which may have a flash memory card related to it. The I/O section is connected to a display, a keyboard, a disk storage unit, and a media drive unit. The media drive unit can read/write a computer-readable medium, which can contain programs and/or data. At least some values based on the results of the processes described herein can be saved for subsequent use. Additionally, a non-transitory computer-readable medium can be used to store (e.g., tangibly embody) one or more computer programs for performing any one of the above-described processes by means of a computer. The computer program may be written, for example, in a general-purpose programming language (e.g., Pascal, C, C++, Java, Python, JSON, etc.) or some specialized application-specific language.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first cancer therapy that exhibit a pathological complete response; receiving or generating a proportion of patients in a second patient population receiving a second cancer therapy that exhibit the pathological complete response; receiving or generating a patient level effect; receiving or generating a residual trial level effect; and determining a trial level hazard ratio for long-term response between the first cancer therapy and the second cancer therapy based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant therapy that exhibit a pathological complete response; receiving or generating a proportion of patients in a second patient population receiving a second neoadjuvant therapy that exhibit the pathological complete response; receiving or generating a patient level effect; receiving or generating a residual trial level effect; and determining a trial level hazard ratio for long-term response between the first neoadjuvant therapy and the second neoadjuvant therapy based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant cancer therapy that exhibit a pathological complete response; receiving or generating a proportion of patients in a second patient population receiving a second neoadjuvant cancer therapy that exhibit the pathological complete response; receiving or generating a patient level effect; receiving or generating a residual trial level effect; and determining a trial level hazard ratio for long-term response between the first neoadjuvant cancer therapy and the second neoadjuvant cancer therapy based on the proportion of patients in the first population of patients that exhibit the pathological complete response, the proportion of patients in the second population of patients that exhibit the pathological complete response, the patient level effect, and the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first cancer therapy that exhibit a pathological complete response; receiving or generating a proportion of patients in a second patient population receiving a second cancer therapy that exhibit the pathological complete response; receiving or generating a patient level effect; receiving or generating a residual trial level effect; and determining a trial level hazard ratio for long-term response between the first cancer therapy and the second cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant therapy that exhibit a pathological complete response; receiving or generating a proportion of patients in a second patient population receiving a second neoadjuvant therapy that exhibit the pathological complete response; receiving or generating a patient level effect; receiving or generating a residual trial level effect; and determining a trial level hazard ratio for long-term response between the first neoadjuvant therapy and the second neoadjuvant therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant cancer therapy that exhibit a pathological complete response; receiving or generating a proportion of patients in a second patient population receiving a second neoadjuvant cancer therapy that exhibit the pathological complete response; receiving or generating a patient level effect; receiving or generating a residual trial level effect; and determining a trial level hazard ratio for long-term response between the first neoadjuvant cancer therapy and the second neoadjuvant cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first cancer therapy that exhibit a pathological complete response; receiving or generating a proportion of patients in a second patient population receiving a second cancer therapy that exhibit the pathological complete response; receiving or generating a patient level effect; receiving or generating a residual trial level effect; and determining a trial level hazard ratio for long-term response between the first cancer therapy and the second cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant therapy that exhibit a pathological complete response; receiving or generating a proportion of patients in a second patient population receiving a second neoadjuvant therapy that exhibit the pathological complete response; receiving or generating a patient level effect; receiving or generating a residual trial level effect; and determining a trial level hazard ratio for long-term response between the first neoadjuvant therapy and the second neoadjuvant therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant cancer therapy that exhibit a pathological complete response; receiving or generating a proportion of patients in a second patient population receiving a second neoadjuvant cancer therapy that exhibit the pathological complete response; receiving or generating a patient level effect; receiving or generating a residual trial level effect; and determining a trial level hazard ratio for long-term response between the first neoadjuvant cancer therapy and the second neoadjuvant cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect or the residual trial level effect are determined from a plurality of historical clinical trials. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first therapy that exhibit a pathological complete response; receiving or generating a proportion of patients in a second patient population receiving a second therapy that exhibit the pathological complete response; receiving or generating a patient level effect; receiving or generating a residual trial level effect; and determining a trial level hazard ratio for long-term response between the first therapy and the second therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third therapy and a fourth therapy for a long-term response for a given historical clinical trial, i; e^{β} is the patient level effect; π_{i,1} is the proportion of patients in receiving the third therapy that exhibit a pathological complete response in the given historical trial, i; and π_{i,0} is the proportion of patients in receiving the fourth therapy that exhibit the pathological complete response in the given historical trial, i. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first cancer therapy that exhibit a pathological complete response; receiving or generating a proportion of patients in a second patient population receiving a second cancer therapy that exhibit the pathological complete response; receiving or generating a patient level effect; receiving or generating a residual trial level effect; and determining a trial level hazard ratio for long-term response between the first cancer therapy and the second cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third cancer therapy and a fourth cancer therapy for a long-term response for a given historical clinical trial, i; e^{β} is the patient level effect; π_{i,1} is the proportion of patients in receiving the third cancer therapy that exhibit a pathological complete response in the given historical trial, i; and π_{i,0} is the proportion of patients in receiving the fourth cancer therapy that exhibit the pathological complete response in the given historical trial, i. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant therapy that exhibit a pathological complete response; receiving or generating a proportion of patients in a second patient population receiving a second neoadjuvant therapy that exhibit the pathological complete response; receiving or generating a patient level effect; receiving or generating a residual trial level effect; and determining a trial level hazard ratio for long-term response between the first neoadjuvant therapy and the second neoadjuvant therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third neoadjuvant therapy and a fourth neoadjuvant therapy for a long-term response for a given historical clinical trial, i; e^{β} is the patient level effect; π_{i,1} is the proportion of patients in receiving the third neoadjuvant therapy that exhibit a pathological complete response in the given historical trial, i; and π_{i,0} is the proportion of patients in receiving the fourth neoadjuvant therapy that exhibit the pathological complete response in the given historical trial, i. In some embodiments, the long-term response is event free survival. In some embodiments, the long-term response is overall survival.

In some embodiments, there is provided a system comprising one or more processors; memory; and one or more programs, wherein the one or more programs are stored in the memory and configured to be executed by the one or more processors, the one or more programs including instructions for receiving or generating a proportion of patients in a first patient population receiving a first neoadjuvant cancer therapy that exhibit a pathological complete response; receiving or generating a proportion of patients in a second patient population receiving a second neoadjuvant cancer therapy that exhibit the pathological complete response; receiving or generating a patient level effect; receiving or generating a residual trial level effect; and determining a trial level hazard ratio for long-term response between the first neoadjuvant cancer therapy and the second neoadjuvant cancer therapy by:

$$λ = \frac{1 + ( {e}^{β} - 1 ) {π}_{1}}{1 + ( {e}^{β} - 1 ) {π}_{0}} {e}^{α} ,$$

wherein λ is the trial level hazard ratio for long-term response; e^{β} is the patient level effect; π₁ is the proportion of patients in the first patient population receiving the first therapy that exhibit the pathological complete response; π₀ is the proportion of patients in the second patient population receiving the second therapy that exhibit the pathological complete response; and e^{α} is the residual trial level effect, wherein the patient level effect and the residual trial level effect are determined from a plurality of historical clinical trials; and wherein the residual trial level effect, e^{α}, is determined, for K historical clinical trials, by:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third neoadjuvant cancer therapy and a fourth neoadjuvant cancer therapy for a long-term response for a given historical clinical trial, i; e^{β} is the patient level effect; π_{i,1} is the proportion of patients in receiving the third neoadjuvant cancer therapy that exhibit a pathological complete response in the given historical trial, i; and π_{i,0} is the proportion of patients in receiving the fourth therapy that exhibit the pathological complete response in the given historical trial, i. In some embodiments, the long-term response is event free survival. In some embodiments, the long-term response is overall survival.

### Determination of the Trial-Level Hazard Ratio

In some embodiments of the methods described herein, a trial-level hazard ratio is determined based on the proportion of patients in the first population of patients that exhibit the pathological complete response, the proportion of patients in the second population of patients that exhibit the pathological complete response, the patient level effect, and the residual trial level effect.

The patient level effect is the hazard ratio of the long-term response between those patients exhibiting a pathological complete response and those patients not exhibiting a pathological complete response, and can be empirically determined from one or more historical clinical trials, as described in further detail herein. The patient level effect converges for a given common identifier of the population of patients. The common identifier can be, for example, a particular disease, such as a particular cancer (e.g., type of cancer (breast cancer, prostate cancer, etc.), biological subtype (e.g., HER2+ breast cancer, triple negative breast cancer, etc.), tumor stage, nodal stage etc.)

The residual trial level effect is any residual effect beyond the prognosis of pathological complete response on the hazard ratio between any two therapies, and can be empirically determined from one or more historical clinical trials, as described in further detail herein. The residual trial level effect converges for a given common identifier of the population of patients (e.g., all of the patients being studies have cancer, all of the patients being studied have breast cancer, all of the patients being studied have HER2-negative breast cancer, etc.).

In some embodiments, the long-term response is overall survival, event free survival, disease free survival, or progression free survival. Event free survival and overall survival are generally preferred long-term response metrics in neoadjuvant therapies.

The pathological complete response is determined after administration of the therapy. The definition of pathological complete response for the first population of patients receiving the first therapy and the definition of pathological complete response for the second population of patients receiving the second therapy are consistent. However, the definition of pathological complete response in a given historical clinical trials need not be consistent with the definition of pathological complete response in every other given historical clinical trial, so long as the definitions share some common feature. That is, a plurality of historical clinical trials can be relied upon even though the historical clinical trials use a different stringent definition of pathological complete response as long as the definitions of pathological complete response share a common feature. For example, if a first historical clinical trial defines pathological complete response as an absence of residual cancer in the breast and regional lymph node and a second historical clinical trial defines pathological complete response as an absence of residual cancer in the breast, both the first historical clinical trial and the second historical clinical trial can be used because both include an absence of residual cancer in the breast as a common feature of the definition of pathological complete response. Nevertheless, in some embodiments the definition of pathological complete response is consistent among the one or more historical clinical trials.

In some embodiments, a hazard ratio (HR) for long-term response between the first therapy (which can be, for example, a test therapy) and a second therapy (such as a control therapy) is determined. A comparison of two patient populations can be used to determine the hazard ratio. The two patient populations have a common identifier, which references the hazard ratio. By way of example and without limiting the scope of the presently disclosed methods, both the first patient population and the second patient population can have breast cancer. Other common identifiers are descried herein.

Preferably, the patients with the common identifier are randomly placed in either the first patient population or the second patient population. Z can be used to denote the treatment assignment for a given individual patient in either patient population, with Z=1 representing the administration of the first therapy and Z=1 representing administration of the second therapy, τ can be used denote the time from the completion of the first therapy to the second therapy to administration of a common second phase therapy (such as surgery or radiation treatment), for example when the first therapy and the second therapy are neoadjuvant therapies. In some embodiments, the second phase therapy is scheduled for a fixed time following the first therapy or the second therapy. In some embodiments, the second phase therapy and the first therapy or the second therapy are administered concurrently. Either way, the time difference between the first therapy or the second therapy and the second phase therapy can be assumed constant. In some embodiments, τ is assumed to be zero. This assumption is proper because the time from administration of the first therapy or the second therapy to the time of long-term response is negligible in most embodiments. R can be used to denote the binary endpoint for pathological complete response, where R=1 means that a given individual in either patient population has a pathological complete response and R=0 means that a given individual in either patient population does not have a pathological complete response.

A hazard function for the hazard ratio of the long-term response at time T (for example, time to disease progression or patient death) between a patient population receiving the first therapy (i.e., Z=1) and a patient population receiving the second therapy (i.e., Z=0) can be defined as:

λ_{(Z,R,τ})(t)=e^{αZ+βR1(t>τ)+γZR1(t>τ)}h₀(t)

for some unknown parameters α, β, γ and baseline hazard function h₀(t). The hazard ratio can also be determined for a given R and τ using the formula:

$${λ}_{( R , τ )} ( t ) = [ \frac{1}{1 + ρ} + \frac{ρ}{1 + ρ} {e}^{α + γ R ( 1 > τ )} ] {e}^{β R 1 ( t > τ )} {h}_{0} ( t ) ,$$

wherein ρ is the randomization ratio. In some embodiments, it is assumed that the randomization ratio is ρ:1 for the patient populations receiving the first therapy and the second therapy. The hazard ratio can further be determined for a given Z and τ using the formula:

λ_{(Z,τ)}(t)=[1−π_{z}+π_{z}e^{(β+γZ)(1>τ)}]e^{αZ}h₀(t).

γ reflects the interaction between assignment to the first patient population or the second patient population and the pathological complete response, and can be determined from one or more historical clinical trials. That is, when γ is not zero, the prognosis of the pathological complete response would be different in patients treated in the first patient population and the second patient population. In some embodiments, γ is assumed to be zero.

β reflects the effect of pathogenic complete response on the hazard ratio between the patient population receiving the first therapy and the patient population receiving the second therapy, and can be determined from the one or more historical clinical trials. The hazard ratio of the long-term response between those patients exhibiting a pathological complete response and those patients not exhibiting a pathological complete response, μ, is referred to as the patient level effect, and is defined as:

$$μ = \frac{1 + {e}^{α + γ}}{1 + {e}^{α}} {e}^{β} .$$

When γ is zero, for example, under the assumption that there is no interaction between the treatment group and the pathological complete response, the patient level effect is reduced to:

μ=e^{β}.

The patient level effect can be determined empirically from one or more historical clinical trials. In some embodiments, the patient level effect is determined using a Cox proportional hazards model, for example based on the pathological complete response association with long-term response of the one or more prior historical clinical trials. In some embodiment, the patient level effect is taken from results reported in the one or more historical clinical trials. In some embodiments, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 15 or more, 17 or more, 18 or more, 19 or more, or 20 or more historical clinical trials are used to determine the patient level effect.

The residual effect beyond the prognosis of pathological complete response on the hazard ratio is referred to as the residual trial level effect, and is reflected by the term:

e^{α}

The residual trial level effect can be determined from the one or more historical clinical trials, for example as described herein.

From the hazard function and assuming τ to be zero, the trial hazard ratio for long-term response between the first therapy (i.e., Z=1) and a second therapy (i.e., Z=0) is determined to be:

$$λ = \frac{1 + ( {e}^{β} - 1 ) {π}_{1}}{1 + ( {e}^{β} - 1 ) {π}_{0}} {e}^{α} .$$

Solving for α results in:

$$α = ln λ - ln \frac{1 + ( {e}^{β} - 1 ) {π}_{1}}{1 + ( {e}^{β} - 1 ) {π}_{0}} .$$

π_{z} reflects the probability of having a pathological complete response given a therapy z, and is defined by:

P(R=1|Z=z)=π_{z}.

That is, π₁ represents the proportion of patients in the first patient population (those receiving the first treatment therapy (Z=1)) that exhibit a pathological complete response (R=1). Similarly, π₀ represents the proportion of patients in the second patient population (those receiving the second treatment therapy (Z=0)) that exhibit a pathological complete response (R=1).

The residual trial level effect can be empirically determined through one or more historical clinical trials by taking the numerical mean of αᵢ from K historical clinical trials, using the formula described above. That is:

$$α = \frac{1}{K} {\sum }_{i = 1}^{K} {α}_{i} ,$$

wherein:

$${α}_{i} = ln {λ}_{i} - ln \frac{1 + ( {e}^{β} - 1 ) {π}_{i , 1}}{1 + ( {e}^{β} - 1 ) {π}_{i , 0}} ,$$

wherein λᵢ is the hazard ratio between a third therapy and a fourth therapy for a long-term response for a given historical clinical trial, i; e^{β} is the patient level effect; Tπ_{i,1} is the proportion of patients in receiving the third therapy that exhibit pathological complete response in the given historical trial, i; and π_{i,0} is the proportion of patients in receiving the fourth therapy that exhibit pathological complete response in the given historical trial, i. The patient level effect, e^{β}, can be determined from the same or other historical clinical trials, as described above.

In some embodiments, the residual trial effect is empirically determined through one or more historical clinical trial by taking the numerical mean of αᵢ from K historical clinical trials, using a patient level effect for the given historical trial rather than a pooled patient level effect. That is, e^{β}^{ᵢ}, wherein i is the given historical trial, rather than e^{β}. Thus, in some embodiments, the residual trial level effect is determined by:

$$α = \frac{1}{K} {\sum }_{i = 1}^{K} {α}_{i} ,$$

wherein:

$${α}_{i} = ln {λ}_{i} - ln \frac{1 + ( {e}^{{β}_{i}} - 1 ) {π}_{i , 1}}{1 + ( {e}^{{β}_{i}} - 1 ) {π}_{i , 0}} ,$$

wherein λᵢ is the hazard ratio between a third therapy and a fourth therapy for a long-term response for a given historical clinical trial, i; e^{β}^{ᵢ} is the patient level effect for the given historical trial; π_{i,1} is the proportion of patients in receiving the third therapy that exhibit pathological complete response in the given historical trial, i; and π_{i,0} is the proportion of patients in receiving the fourth therapy that exhibit pathological complete response in the given historical trial, i.

In some embodiments, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 15 or more, 17 or more, 18 or more, 19 or more, or 20 or more historical clinical trials are used to determine the residual trial level effect.

In some embodiments, the one or more historical clinical trials include AGO 1 (Untch et al., J. Clin. Oncol., vol. 27, pp. 2938-2945 (2009)), ECTO (Gianni et al., J. Clin. Oncol., vol. 27, pp. 2474-2481 (2009)), EORTC 10994/BIG 1-00 (Bonnefoi et al., Lancet Oncol., vol. 12, pp. 527-539 (2011)), GeparDuo (von Minckwitz et al., J. Clin. Oncol., vol. 23, pp. 2676-2685 (2005)). GeparQuattro (von Mickwitz et al., J. Clin. Oncol., vol. 28, pp. 2024-2031 (2010); Untch et al., J. Clin. Oncol., vol. 28. pp. 2024-2031 (2010)). GeparTrio (von Mickwitz et al., J. Nat'l Cancer Inst., vol. 100. pp. 542-551 (2008); von Minckwitz. J. nat'l Cancer Inst., vol. 100, pp. 552-562 (2008)), GepartTrio-Pilot (von Mickwitz, Ann. Oncol., vol. 16, pp. 56-63 (2005)), NOAH (Gianni et al., Lancet, vol. 375, pp. 377-384 (2010)), NSABP B-18 (Wolmark et al., J. Nat'l Cancer Inst. Monogr., vol. 30, pp. 96-102 (2001); Rastogi et al., J. Clin. Oncol., vol. 26, pp. 778-785 (2008)), NSABP B-27 (Rastogi et al., J. Clin. Oncol., vol. 26, pp. 778-785 (2008); Bear et al., J. Clin. Oncol., vol. 21, pp. 4165-4174 (2003)), PREPARE (Untch et al., Ann. Oncol., vol. 22. pp. 1988-1998 (2011); Untch et al., Ann. Oncol., vol. 22. pp. 1999-2006 (2011)), or TECHNO (Untch et al., J. Clin. Oncol., vol. 29, pp. 3351-3352 (2011)).

### Example

A trial-level hazard ratio for event free survival (EFS) and for overall survival (OS) was determined for the GeparSixto study (von Minckwitz et al., Neoadjuvant carboplatin in patients with triple-negative and HER2-positive early breast cancer (GeparSixto; GBG 66): a randomize phase 2 trial. Lancet Oncology, vol. 15. pp. 747-756 (2014)) by re-analyzing the data presented in Cortazar et al., Lancet, vol. 384, pp. 164-172 (2014) in accordance with the methods described herein. The GeparSixto study was randomized phase 2 clinical trial comparing a first neoadjuvant chemotherapy of carboplatin, paclitaxel, and non-pegylated liposomal doxorubicin administered to a first patient population (n=295) to a second neoadjuvant chemotherapy of paclitaxel and non-pegylated liposomal doxorubicin administered to a second patient population (n=293) prior to surgery for triple-negative breast cancer and HER2-positive breast cancer patients.

The results of the GeparSixto study showed that the proportion of patients in the first patient population exhibiting pathogenic complete response (defined by ypT0/is ypN0). i.e., π₁, was 53.2%. The proportion of patients in the second patient population exhibiting pathogenic complete response (defined by ypT0/is ypN0), i.e., π₀, was 47.4%. The hazard ratio disease free survival (DFS) was reported as 0.81, with a 95% confidence interval of (0.54, 1.21) for the GeparSixto study.

Cortazar et al. reported a meta-analysis from 12 international breast cancer adjuvant therapy trials for 11,955 patients. The patient level effect (e^{β}) and the residual trial level effect (α) were determined using the data presented in Cortazar et al. and the clinical studies cited therein.

The hazard ratio for event free survival between pathological complete response and non pathological complete response for the long-term response (i.e., the patient level effect, e^{β}) was obtained from FIG. 2 of Cortazar et al. Cortazar et al. pooled the results of several studies comparing a first neoadjuvant breast cancer therapy and a second neoadjuvant breast cancer therapy to determine a patient level effect for long-term response based on a definition of pathological complete response of ypT0/is ypN0 and was calculated with a Cox proportional hazard model. Cortazar et al. reported a hazard ratio between pathological complete response and non pathological complete response for event free survival (e^{β}) of 0.48 with a 95% confidence interval of (0.43, 0.54), and a hazard ratio between pathological complete response and non pathological complete response for overall survival (e^{β}) of 0.36 (with a 95% confidence interval of 0.31-0.42).

The residual trial level effect (α) for the long-term response was determined by re-analyzing data from the studies analyzed in Cortazar et al. Table 1 presents the proportion of patients in a first patient population receiving a first neoadjuvant breast cancer therapy exhibiting a pathological complete response (π_{i,1}) and the proportion of patients in a second patient population receiving a second neoadjuvant breast cancer therapy exhibiting a pathological complete response (π_{i,0}), and the hazard ratio for long term response (λᵢ) for each given trial. The patient level effect (e^{β}) reported Cortazar et al., was used to determine the residual trial level effect for each given study (αᵢ) was determined using the formula:

$${α}_{i} = ln {λ}_{i} - ln \frac{1 + ( {e}^{β} - 1 ) {π}_{i , 1}}{1 + ( {e}^{β} - 1 ) {π}_{i , 0}} .$$

TABLE 1

| | | | e^{β} | e^{β} | λᵢ | λᵢ | αᵢ | αᵢ |
|---|---|---|---|---|---|---|---|---|
| Study | π_{i, 0} | π_{i, 1} | (EFS) | (OS) | (EFS) | (OS) | (EFS) | (OS) |
| GeparQuattro_a | 0.22290 | 0.22340 | 0.480 | 0.360 | 1.0472 | 0.8105 | 0.046 | −0.210 |
| GeparDuo | 0.06874 | 0.13907 | 0.480 | 0.360 | 1.1153 | 1.0186 | 0.148 | 0.067 |
| GeparQuattro_c | 0.22290 | 0.19530 | 0.480 | 0.360 | 0.8795 | 0.8396 | −0.145 | −0.195 |
| EORTC10994 | 0.23500 | 0.26500 | 0.480 | 0.360 | 0.8500 | 0.8900 | −0.145 | −0.094 |
| PREPARE | 0.14300 | 0.20900 | 0.480 | 0.360 | 0.8688 | 0.7994 | −0.047 | 0.007 |
| NSABP-B27 | 0.13700 | 0.26100 | 0.480 | 0.360 | 0.8877 | 0.9192 | −0.274 | −0.255 |
| GeparTrio Resp | 0.21020 | 0.23500 | 0.480 | 0.360 | 0.7494 | 0.7609 | −0.374 | −0.203 |
| GeparTrio NonResp | 0.05980 | 0.05296 | 0.480 | 0.360 | 0.6906 | 0.8203 | −0.310 | −0.146 |
| AGO1 | 0.06000 | 0.12000 | 0.480 | 0.360 | 0.7100 | 0.8300 | −0.412 | −0.362 |
| NOAH | 0.19000 | 0.03800 | 0.480 | 0.360 | 0.5900 | 0.5999 | 0.046 | −0.210 |

A Kolmogorv-Smirnov test for normal distribution of residual trial level effect for each given historical clinical study (αᵢ) for event free survival yielded a p-value greater than 0.15, suggesting no lack of fit. A Student's t test for non-zero central location of the calculated residual trial level effects for given historical clinical studies (αᵢ) resulted in a p-value of 0.0206. The mean residual trial level effect (α) was calculated as −0.16139. Using the formula

$${λ}_{EFS} = \frac{1 + ( {e}^{β} - 1 ) {π}_{1}}{1 + ( {e}^{β} - 1 ) {π}_{0}} {e}^{α} ,$$

the trial level hazard ratio for event free survival between the first therapy and the second therapy, based on π₁ and π₀ reported in GeparSixto study and the residual trial level effect and the patient level effect determined above, was determined to be 0.8169. This compares closely with the reported hazard ratio for disease free survival between the first therapy and the second therapy determined from the GeparSixto study of 0.81.

A Kolmogorv-Smirnov test for normal distribution of residual trial level effect for each given historical clinical study (αᵢ) for overall survival yielded a p-value greater than 0.15, suggesting no lack of fit. A Student's t test for non-zero central location of the calculated residual trial level effects for given historical clinical studies (αᵢ) resulted in a p-value of 0.0032. The mean residual trial level effect (α) was calculated as −0.15664. Using the formula

$${λ}_{OS} = \frac{1 + ( {e}^{β} - 1 ) {π}_{1}}{1 + ( {e}^{β} - 1 ) {π}_{0}} {e}^{α} ,$$

the trial level hazard ratio for overall survival between the first therapy and the second therapy, based on π₁ and π₀ reported in the GeparSixto study and the residual trial level effect and the patient level effect determined above, was determined to be 0.80945.

To validate the predicted hazard ratio from the empirical predictive model, the event free survival or disease free survival data from GeparSixto, BOOG (Vriens et al., Doxorubicin/cyclophosphamide with concurrent versus sequential docetarel as neoadjuvant treatment in patients with breast cancer. Eur J Cancer, vol. 49, pp. 3102-3110 (2013); and Vriens et al., Doxorubicin/cyclophosphamide with concurrent versus sequential docetaxel as neoadjuvant treatment in patients with breast cancer—5-year disease-free and overall survival data. Presented at: European Breast Cancer Conference; Mar. 9-11, 2016; Amsterdam, the Netherlands), and Neo-tAnGo (Earl et al., Effects of the addition of gemcitabine, and paclitaxel-first sequencing, in neoadjuvant sequential epirubicin, cyclophosphamide, and paclitaxel for women with high-risk early breast cancer (Neo-tAnGo): an open-label, 2×2 factorial randomised phase 3 trial. Lancet Oncolology, vol. 15, pp. 201-201 (2014)) studies were used as the validation set. The observed hazard ratios for event free survival and disease free survival are provided in Table 2 in the column “Observed HR.” The predicted hazard ratio derived using the predictive formula is provided in the column “Predicted HR.” The predictive accuracy of a model is by the mean squared error (MSE) on the validation set. It is calculated as follows, and the related results are provided in Table 2. The log(observed HR) is set to y, and the log (predicted HR) is set to x. ŷ is determined based on the regression line, where ŷ=1.029*x. The MSE for the validation set based on y, ŷ, and the number of validation data sets (n=3 in this Example) is determined as follows:

$$MSE for validation set = \frac{\sum {( \hat{y} - y )}^{2}}{n . validation - 1} = 0.1088$$

The training set MSE is 0.0331, obtained from the model-fit output of the training-set regression model. The ratio of the 2 MSEs is 0.1088/0.0331=3.287. The resulting P value from the F test is 0.085, which does not suggest statistical lack of fit for the validation data set.

TABLE 2
Data for Validation Set

| | | | y = log | x = log | |
|---|---|---|---|---|---|
| | Observed | Predicted | (observed | (predicted | ŷ = |
| Studies | HR | HR | HR) | HR) | 1.029*x |
| GeparSixto | 0.81 | 0.82 | −0.211 | −0.204 | −0.210 |
| BOOG | 0.53 | 0.83 | −0.635 | −0.192 | −0.198 |
| Neo-tAnGo | 0.82 | 0.70 | −0.198 | −0.350 | −0.360 |

HR, hazard ratio.

1: A method of treating an individual patient comprising administering a first therapy to the individual patient if a trial level hazard ratio for long-term response between the first therapy and a second therapy is below a predetermined threshold;
wherein the trial level hazard ratio is determined by:
a) obtaining a proportion of patients in a first population of patients receiving the first therapy that exhibit a pathological complete response;
b) obtaining a proportion of patients in a second population of patients receiving the second therapy that exhibit the pathological complete response;
c) obtaining a patient level effect and a residual trial level effect; and
d) determining the trial level hazard ratio based on the proportion of patients in the first population of patients that exhibit the pathological complete response, the proportion of patients in the second population of patients that exhibit the pathological complete response, the patient level effect, and the residual trial level effect.

2: A method of conducting a therapy trial comprising:
a) obtaining a proportion of patients in a first patient population receiving a first therapy that exhibit a pathological complete response;
b) obtaining a proportion of patients in a second patient population receiving a second therapy that exhibit the pathological complete response;
c) obtaining a patient level effect and a residual trial level effect;
d) determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect; and
e) administering the first therapy to a third patient population if the trial level hazard ratio is below a predetermined threshold.

3: The method of claim 1, wherein the first therapy and the second therapy are neoadjuvant therapies.

4: The method of claim 1, wherein the trial level hazard ratio is determined by:
$$λ = \frac{1 + ( {e}^{β} - 1 ) {π}_{1}}{1 + ( {e}^{β} - 1 ) {π}_{0}} {e}^{α} ,$$
wherein:
λ is the trial level hazard ratio for long-term response;
e^{β} is the patient level effect;
π₁ is the proportion of patients in the first patient population receiving the first therapy that exhibit the pathological complete response;
π₀ is the proportion of patients in the second patient population receiving the second therapy that exhibit the pathological complete response; and
e^{α} is the residual trial level effect.

5. (canceled)

6: The method of claim 1, wherein the patient level effect or the residual trial level effect are determined from a plurality of historical clinical trials.

7: The method of claim 1, wherein the patient level effect is based on a hazard ratio between pathological complete response and non pathological complete response for the long-term response in the plurality of historical clinical trials.

8: The method of claim 7, wherein the patient level effect is determined using a Cox proportional hazards model.

9: The method of claim 6, wherein the residual trial level effect, e^{α}, is determined, for K historical clinical trials, by:
$$α = \frac{1}{K} {\sum }_{i = 1}^{K} {α}_{i} ,$$
wherein:
$${α}_{i} = ln {λ}_{i} - ln \frac{1 + ( {e}^{β} - 1 ) {π}_{i , 1}}{1 + ( {e}^{β} - 1 ) {π}_{i , 0}} ,$$
wherein:
λᵢ is a hazard ratio between a third therapy and a fourth therapy for a long-term response for a given historical clinical trial, i;
e^{β} is the patient level effect;
π_{i,1} is a proportion of patients in receiving the third therapy that exhibit a pathological complete response in the given historical trial, i; and
π_{i,0} is a proportion of patients in receiving the fourth therapy that exhibit the pathological complete response in the given historical trial, i.

10: The method of claim 6, wherein the residual trial level effect, e^{α}, is determined, for K historical clinical trials, by:
$$α = \frac{1}{K} {\sum }_{i = 1}^{K} {α}_{i} ,$$
wherein:
$${α}_{i} = ln {λ}_{i} - ln \frac{1 + ( {e}^{{β}_{i}} - 1 ) {π}_{i , 1}}{1 + ( ( {e}^{{β}_{i}} - 1 ) {π}_{i , 0}} ,$$
wherein:
λᵢ is a hazard ratio between a third therapy and a fourth therapy for a long-term response for a given historical clinical trial, i;
e^{β}^{ᵢ} is a patient level effect for a given historical clinical trial, i;
π_{i,1} is a proportion of patients in receiving the third therapy that exhibit a pathological complete response in the given historical trial, i; and
π_{i,0} is a proportion of patients in receiving the fourth therapy that exhibit the pathological complete response in the given historical trial, i.

11: The method of claim 9, wherein the third therapy and the fourth therapy are neoadjuvant therapies.

12: The method of claim 1, wherein the long-term response is event free survival.

13: The method of claim 1, wherein the long-term response is overall survival.

14: The method of claim 1, wherein the first therapy and the second therapy are cancer therapies.

15: The method of claim 1, wherein the first population of patients and the second population of patients have breast cancer.

16: The method of claim 1, wherein the first population of patients and the second population of patients have HER2− breast cancer.

17: The method of claim 1, wherein the first population of patients and the second population of patients have triple negative breast cancer.

18: The method of claim 1, wherein the first population of patients and the second population of patients have HER2+ breast cancer.

19: The method of claim 1, wherein the pathological complete response is ypT0 ypN0 or ypT0/is ypN0.

20: The method of claim 1, wherein the first therapy and the second therapy are breast cancer therapies.

21: The method of claim 1, wherein the first therapy or the second therapy comprises administration of a taxane.

22: The method of claim 1, wherein the first therapy and the second therapy are followed by surgery or radiation treatment.

23: The method of claim 22, wherein the pathological complete response is determined at about the same time as a surgery or radiation treatment.

24: A system comprising:
one or more processors;
memory; and
one or more programs, wherein the one or more programs are stored in the memory and configured to be executed by the one or more processors, the one or more programs including instructions for:
a) receiving or generating a proportion of patients in a first patient population receiving a first therapy that exhibit a pathological complete response;
b) receiving or generating a proportion of patients in a second patient population receiving a second therapy that exhibit the pathological complete response;
c) receiving or generating a patient level effect;
d) receiving or generating a residual trial level effect; and
e) determining a trial level hazard ratio for long-term response between the first therapy and the second therapy based on the proportion of patients in the first population of patients that exhibit the pathological complete response, the proportion of patients in the second population of patients that exhibit the pathological complete response, the patient level effect, and the residual trial level effect.
